(19) United States
(12) Patent Application Publication
(10) Pub. No.: US 2008/0305470 A1
(43) Pub. Date: Dec. 11, 2008

(54) Nucleic Acid Sequencing

(75) Inventor: Frank Larsen, Oslo (NO)

Correspondence Address: HENRY M FEIEREISEN, LLC;HENRY M FEIEREISEN, 708 THIRD AVENUE, SUITE 1501, NEW YORK, NY, 10017, US

(73) Assignee: QIAGEN AS, Oslo (NO)

(21) Appl. No.: 10/599,349

(22) PCT Filed: Mar. 24, 2005
(86) PCT No.: PCT/IB2005/000761

§ 371 Date: Aug. 22, 2008

(30) Foreign Application Priority Data
Mar. 26, 2004 (GB) 0406865.6

(51) Int. Cl. C12Q 1/68

(52) U.S. Cl. 435/6

(57) ABSTRACT

A method for determining a target nucleic acid sequence, wherein the target nucleic acid sequence is comprised in a preparation comprising a non-target nucleic acid sequence, the target nucleic acid sequence and the non-target nucleic acid sequence each having a first region of common sequence upstream of a first region of dissimilar sequence upstream of a second region of dissimilar sequence, the method comprising:
(a) contacting the preparation with an oligonucleotide primer complementary to at least a portion of the first region of common sequence, under conditions to hybridise the primer thereto; and
(b) subjecting the preparation to a sequencing reaction, such that the sequencing reaction proceeds into the second region of dissimilar sequence of the target nucleic acid sequence, thereby determining at least the second region of dissimilar sequence of the target nucleic acid sequence;
   and wherein the method further comprises a step of blocking the sequencing reaction between the primer and the non-target nucleic acid sequence, such that the sequencing reaction does not proceed into the second region of dissimilar sequence of the non-target nucleic acid sequence.

The present invention relates to a method of sequencing a nucleic acid. In particular, the invention relates to a method for determining a target nucleic acid sequence where the target nucleic acid sequence is comprised in a preparation comprising a non-target nucleic acid sequence. The invention also relates to a method for determining the haplotype of a subject.

The sequencing of nucleic acids, in particular DNA, is of fundamental importance in many areas of biological research, clinical diagnosis and treatment. Sequencing of DNA is typically carried out by a method based on the Sanger dideoxy chain-termination method (Sanger, F., Nicklen, S., and Coulson, A. R. (1977) “DNA Sequencing with chain-terminating inhibitors” PNAS USA 74:5463-5467). In this method, a labelled oligonucleotide primer complementary to a known sequence adjacent to the target sequence is used to initiate DNA polymerase-catalysed elongation into the target sequence. Typically, four polymerase reactions are carried out for each round of sequencing. Each reaction contains all four deoxynucleotides (dNTPs-dCTP, dTTP, dGTP and dATP) plus a small amount of one dideoxynucleotide (ddNTP-ddCTP, ddTTP, ddGTP or ddATP). Because ddNTPs have no 3′ hydroxyl group, elongation of the nascent strand is occasionally terminated by incorporation of a ddNTP. Thus the sequencing reaction produces a series of labelled strands whose lengths are indicative of the location of a particular base in the sequence. The resultant labelled strands are typically separated according to size by polyacrylamide gel electrophoresis and visualised by detecting the label, for example by autoradiography where the primer was radiolabelled. More recently, the Sanger sequencing method has been adapted in various ways, in particular for large-scale automated sequencing using multiple fluorescent labels and capillary gel electrophoresis.

One problem with sequencing methods based on the Sanger method occurs when the target nucleic acid to be sequenced is provided in a preparation comprising one or more different nucleic acids or sequences which show some sequence identity to the target sequence. In particular, if a primer-binding sequence is found in both the target sequence and a second or further sequences, the sequencing reaction will lead to products which are derived from primer binding to the second or further sequences, as well as the target sequence. Where the target sequence diverges from the second or further sequences, the resultant gel or chromatograph will reveal two or more bases as being present at a particular location. Because the method does not allow discrimination between the products of the target sequence and the second or further sequences, the target sequence cannot be determined unambiguously.

This problem is particularly significant when it is desired to determine the sequence of one allele of a heterozygote pair at a polymorphic location in a single individual. Many eukaryotic cells are diploid, having two copies of most chromosomes, and sequence differences usually exist between each copy of a particular chromosome. Because DNA prepared from one individual will normally contain copies of both chromosomes, standard sequencing methods are unable to differentiate between sequences derived from each copy. Where there is a single nucleotide difference between each allele, the DNA sequence of each chromosome will nevertheless be clear (although it would not be possible to ascribe each sequence to a particular paternal or maternal chromosome). Where the polymorphism extends for two or more nucleotides, or where there are two or more polymorphic sites (alleles) separated by regions of common sequence, it is not possible to discern the sequence of the two alleles. In particular, standard sequencing methods are not able to determine the combination of alleles existing on a particular chromosome (the haplotype).

In the wave of interest spawned by the mapping of the human genome, interest has grown in the use of single nucleotide polymorphisms (SNPs) to identify target genes associated with disease or drug response. In some instances, the presence of a particular SNP alone may be sufficient to cause a particular disease or to explain the individual variability in sensitivity to drugs.

However, it is not clear how often knowledge of an individual SNP will have utility in the clinic or in drug development. Research has shown that in asthma, at least, the association of individual SNPs to form a complete haplotype may be more relevant in predicting drug response than knowledge of isolated individual SNPs. In many cases it may be necessary to obtain a haplotype sequence involving the characterisation of two or more SNPs on each chromosome. It is therefore highly desirable to determine the combination of SNPs that co-exist on a single chromosome.

HLA (human leukocyte antigen or human leukocyte associated antigen A) genotyping is one area where haplotyping is important. Determination of the two haplotype sequences of the HLA genes is crucial to the success of organ transplantation. The individual haplotypes of the donor must be matched with the recipient before transplantation to avoid rejection of the transplant. Methods for evaluating HLA allele types have been described in the past. One such method relies on performing family studies, which is very time-consuming. An alternative method based on DNA sequencing is disclosed in WO 97/23650. However, where heterozygous alleles exist, this method relies on prior knowledge of existing haplotype sequences, so that ambiguous bases can be ascribed to one allele or another.

Many of the methods used for haplotyping used in the past rely on preparing a composition comprising only a single haplotype sequence before sequencing. One way of doing this is by converting a diploid cell into a haploid cell. This requires a high investment, is labour intensive and slow but gives complete haplotype separation. Alternatively, human chromosomes can be cloned into yeast in order to get a haploid for that particular chromosome. This suffers from the same drawbacks in terms of time and cost.

One way of obtaining a preparation comprising only a single haplotype sequence is to amplify DNA by PCR using allele-specific primers. This type of approach for sequencing both alleles of a deletion polymorphism in intron 6 of the human dopamine 2 receptor gene (DRD2) is described in DNA Sequence Vol 6 (2), pp 87-94 (1996), Finck et al. In this method, allele-specific primers are used to amplify individual allele sequences by polymerase chain reaction (PCR). The primers are designed so that they produce amplicons of differing lengths, so that the products of each allele can be discriminated by agarose gel electrophoresis when both alleles are simultaneously amplified in the same reaction tube. The amplicons from each allele are then extracted from the gel and sequenced using conserved primers. The disadvantage of this approach is that it requires the prior knowledge of at least two, sufficiently separated regions of dissimilarity between the alleles so that appropriate allele-specific primers producing different-sized products can be designed. In addition, it requires a time-consuming gel separation and extraction step prior to sequencing.

A related approach is described in Biotechniques Vol 10 (1), pp 30, 32 and 34 (1991), Kaneoka et al. Biotinylated allele-specific oligonucleotide primers coupled to streptavidin-coated magnetic beads are used to amplify DNA from one haplotype by PCR, and then conserved primer is used for solid-phase direct DNA sequencing.

WO 92/15711 discloses a method for determining a major histocompatibility complex genotype of a subject in a sample containing nucleic acid. The method involves PCR amplification of the gene locus of interest, with all alleles for the gene locus to be sequenced being amplified with one conserved oligonucleotide primer pair and at least one allele for the gene locus being amplified with one conserved oligonucleotide primer and one non-conserved oligonucleotide primer. The amplicons for each allele are then sequenced with a conserved primer.

A different method for determining haplotype sequences involves analysis of PCR amplified sequences covering a polymorphic region by hybridisation rather than sequencing. PCR amplicons are contacted with oligonucleotide probes complementary to the sequence of either the maternal or paternal chromosome in a region comprising an SNP. Probes complementary to the maternal or paternal chromosomes are immobilised in different areas of a solid phase. A second set of oligonucleotide probes, labelled in a different way and complementary to the sequence of either the maternal or paternal chromosome in a region comprising a second SNP, is then used to identify which sequence at the first SNP is on the same chromosome as a particular sequence at the second SNP.

Other approaches have been adopted in the past for determining a target nucleic acid sequence when the target sequence is contained in a preparation comprising a non-target nucleic acid sequence. In one method described in WO 97/46711, a primer is selected that complements one strand but not the other, and an artificial mismatch is introduced into the primer. By selecting suitable hybridisation conditions so that stable duplexes form between the primer and one allele but not between the primer and the other allele, chain-extension sequencing of a single allele is achieved. A disadvantage of this method is that the selection of appropriate hybridisation conditions is time-consuming and not necessarily straightforward.

WO 00/20628 describes a method by which multiple genomic loci can be sequenced in the same reaction mixture. This method allows the sequencing of a second locus in the mixture by using primers which are longer than the longest product formed from the sequencing reaction in relation to a first locus. Different primers are used for each locus. However, this document does not disclose a method for haplotyping for particular alleles of a single locus.

Accordingly, the present invention aims to overcome the disadvantages of the prior art. In particular, the present invention aims to provide an improved method of determining a target nucleic acid sequence, where the target nucleic acid is comprised in a preparation comprising a non-target nucleic acid which has regions of common and dissimilar sequence to the target nucleic acid. The present invention also aims to provide an improved method for determining the haplotype of a subject.

Accordingly, the present invention provides a method for determining a target nucleic acid sequence, wherein the target nucleic acid sequence is comprised in a preparation comprising a non-target nucleic acid sequence, the target nucleic acid sequence and the non-target nucleic acid sequence each having a first region of common sequence upstream of a first region of dissimilar sequence upstream of a second region of dissimilar sequence, the method comprising:

(a) contacting the preparation with an oligonucleotide primer complementary to at least a portion of the first region of common sequence, under conditions to hybridise the primer thereto; and
(b) subjecting the preparation to a sequencing reaction, such that the sequencing reaction proceeds into the second region of dissimilar sequence of the target nucleic acid sequence, thereby determining at least the second region of dissimilar sequence of the target nucleic acid sequence;

and wherein the method further comprises a step of blocking the sequencing reaction between the primer and the non-target nucleic acid sequence, such that the sequencing reaction does not proceed into the second region of dissimilar sequence of the non-target nucleic acid sequence.

In a further aspect, the present invention provides a method for determining the haplotype of a subject from a sample comprising DNA from the subject, comprising a method as defined above, wherein the preparation comprises the sample, the target nucleic acid sequence comprises a locus on a first chromosome of a pair of chromosomes, the non-target nucleic acid sequence comprises the corresponding locus on the second chromosome of the pair, the locus comprising two or more single nucleotide polymorphisms for which the subject is heterozygous, wherein the sequencing reaction is conducted to determine the sequence of the polymorphic genetic locus on the first chromosome of the pair thereby determining the haplotype of the subject.

In a further aspect, the present invention provides use of pyrosequencing for determining the haplotype of a subject from a sample comprising DNA from the subject, wherein pyrosequencing is used to sequence a target locus on a first chromosome of a pair, the target locus comprising two or more single nucleotide polymorphisms, the corresponding locus on the second chromosome of the pair being blocked from sequencing.

The present invention provides an improved method of sequencing a target nucleic acid sequence comprised in a preparation comprising a different but related nucleic acid sequence. The method advantageously allows the sequencing reaction to proceed in relation to the target nucleic acid sequence, while the sequencing reaction between the primer and the other nucleic acid sequence is blocked. The sequence data which is obtained is therefore derived only from the target nucleic acid sequence, as interference from the other nucleic acid sequence is removed. The method is a fast and efficient way of discriminating between the two sequences. In particular, the method is advantageous because a sequence-specific sequencing primer does not have to be constructed for each target nucleic acid sequence. The method also does not suffer from problems relating to lack of discrimination in primer hybridisation to closely-related sequences.

The method also provides an enhanced method for haplotyping. The method enables the rapid determination of allele associations to identify individually the two haplotype sequences present at a particular locus in a subject. The method is particularly advantageous in identifying associations of SNPs and in HLA genotyping. In particular, the method avoids the need for time-consuming family studies or prior knowledge of allele associations.

The target nucleic acid sequence of the present invention is not particularly limited. Suitable target nucleic acid sequences include a deoxyribonucleic acid (DNA) sequence, a ribonucleic acid (RNA) sequence, or a DNA or RNA sequence comprising one or more modified nucleotides or bases, or one or more artificial nucleotides or bases. The non-target nucleic acid sequence is likewise not particularly limited, and may be a DNA or RNA sequence, optionally comprising one or more modified nucleotides or bases.

Preferably the target nucleic acid sequence and/or the non-target nucleic acid sequence is a DNA sequence. The DNA sequence may be a genomic DNA or cDNA sequence. Each sequence is preferably a human DNA sequence.

The target nucleic acid sequence may be comprised in the same nucleic acid polymer as the non-target nucleic acid. However, the two nucleic acid sequences are preferably on separate DNA molecules. More preferably the target nucleic acid sequence and the non-target nucleic acid sequence each comprise one allele at a polymorphic genetic locus in a subject. In this embodiment, the target nucleic acid sequence comprises the locus on one chromosome of a pair (maternal or paternal) and the non-target nucleic acid sequence comprises the locus on the other chromosome of the pair.

In the present invention the preparation comprises a target nucleic acid sequence and a non-target nucleic acid sequence. Suitable preparations include any preparation comprising two or more nucleic acid sequences, provided that at least two of the nucleic acid sequences share a region of common sequence but differ in a region of dissimilar sequence. Preferably the preparation comprises a purified DNA preparation. The preparation is preferably prepared from a sample derived from a single human subject. Thus the preparation may be a sample of human saliva, blood, urine or other tissue, or a DNA preparation comprising genomic DNA which has been prepared from such a sample.

In one embodiment, the preparation comprises one or more further nucleic acid sequences, wherein each further nucleic acid sequence has a first region of common sequence upstream of a first region of dissimilar sequence upstream of a second region of dissimilar sequence. Here “common sequence” means that the sequence of the further nucleic acid sequence is identical to the target and non-target nucleic acid sequences in this region. “Dissimilar sequence” means that the sequence of the further nucleic acid is different from the target and/or non-target nucleic acid sequences in this region.

In this embodiment, the method may include a step of blocking the sequencing reaction between the primer and one or more of the further nucleic acid sequences. The sequencing reaction between the primer and the further nucleic acid sequences may be blocked in the same way as for the sequencing reaction between the primer and the non-target nucleic acid sequence. If it is desired to obtain sequencing reaction products derived only from the target nucleic acid sequence, the sequencing reaction between the primer and each of the further nucleic acid sequences may be blocked.

Alternatively, the sequencing reaction between the primer and only some of the further nucleic acid sequences may be blocked. By using the methods described below, sequencing from particular further nucleic acid sequences may be selectively blocked or allowed to proceed. This type of analysis may be termed “multiplexing”. Multiplexing permits the analysis of multiple sites in an individual sample or a number of samples from different individuals.

In one embodiment using multiplexing, the preparation comprises DNA derived from samples taken from two or more individuals. For instance, a number of DNA preparations derived from different individuals in a group may be combined and the method described herein carried on the combined preparation. This method may be used to assess whether or not a particular combination of SNPs is found together on a single chromosome in all individuals within the group. If so, the sequencing reaction will yield a single sequence. If not, the sequencing reaction will indicate alternative bases at the position of one or more SNPs in the sequence. If it is then desired to determine which combination of SNPs was present in which individual, it would be necessary to repeat the method on separate DNA preparations from each individual.

In another embodiment involving multiplexing, more than one target nucleic acid sequence may be determined using a single sequencing reaction. In this embodiment, the present method is performed in parallel using two or more oligonucleotide primers, each of which is complementary to a different sequence. In this way, two or more polymorphic sites may be analysed simultaneously. Each target nucleic acid sequence shares a first region of common sequence with a corresponding non-target nucleic acid sequence. The sequencing reaction between each primer and the non-target nucleic acid sequence to which it is complementary is blocked, so that the sequencing reaction proceeds fully only in respect of the target nucleic acid sequences.

In one such embodiment, the invention relates to a method for determining a plurality of target nucleic acid sequences, wherein the plurality of target nucleic acid sequences is comprised in a preparation further comprising a plurality of corresponding non-target nucleic acid sequences, each target nucleic acid sequence in the preparation corresponds to one or more corresponding non-target nucleic acid sequences in the preparation, each target nucleic acid sequence and each corresponding non-target nucleic acid sequence has a first region of common sequence upstream of a first region of dissimilar sequence upstream of a second region of dissimilar sequence, the first region of common sequence of each target nucleic acid sequence is the same as the first region of common sequence of its corresponding non-target nucleic acid sequences, the first region of dissimilar sequence of each target nucleic acid sequence is different to the first region of dissimilar sequence of its corresponding non-target nucleic acid sequences, the second region of dissimilar sequence of each target nucleic acid sequence is different to the second region of dissimilar sequence of its corresponding non-target nucleic acid sequences, which method comprises:

(a) contacting the preparation with a plurality of oligonucleotide primers, wherein each primer is complementary to at least a portion of the first region of common sequence of a target nucleic acid sequence and its corresponding non-target nucleic acid sequence, under conditions to hybridise the primer thereto; and
(b) subjecting the preparation to a sequencing reaction, such that the sequencing reaction proceeds into the second region of dissimilar sequence of the target nucleic acid sequences, thereby determining at least the second region of dissimilar sequence of each target nucleic acid sequence;

and wherein the method further comprises a step of blocking the sequencing reaction between each primer and each corresponding non-target nucleic acid sequence, such that the sequencing reaction does not proceed into the second region of dissimilar sequence of each corresponding non-target nucleic acid sequence.

In this embodiment, sequencing reaction products are obtained which are derived from more than one target nucleic acid sequence. A method is therefore required in order to discriminate between sequencing reaction products derived from each target nucleic acid sequence. This may be done by labelling the sequencing reaction products derived from each nucleic acid sequence in which the sequencing reaction is allowed to proceed with a distinct label.

The sequencing reaction products derived from each target nucleic acid sequence may be distinguished by differentially labelling each oligonucleotide primer. In one embodiment, each primer is labelled with a fluorescent label which fluoresces at a different wavelength. Sequencing products derived from each target nucleic acid sequence may then be distinguished for example using an automated sequencer following gel electrophoresis. In another embodiment, one or more primers is labelled with one part of a ligand-affinant pair. A preferred ligand-affinant pair is biotin-streptavidin. The ligand-affinant interaction may be used in order to bind sequencing products derived from one target nucleic acid sequence to a solid phase (such as magnetic beads), thereby separating the labelled sequencing products from non-labelled sequencing products. The labelled and non-labelled sequencing may then be separately subjected to gel electrophoresis. In embodiments where two primers are used (in order to sequence two different target nucleic acid sequences), only one primer need be labelled in order to separate the sequencing products derived from each of the target nucleic acid sequences. In embodiments where 3 or more primers are used, 2 or more of the primers need to be labelled. In this case, a different ligand-affinant pair needs to be selected for each primer to be labelled, so that the sequencing products derived from each target nucleic acid sequence can be bound to a different solid phase and thereby separated. In general, where n primers are used, n−1 primers need to be labelled.

Each of the two nucleic acid sequences includes a first region of common sequence. This means that the target nucleic acid sequence is identical to the non-target nucleic acid sequence in this region. The method advantageously allows the sequencing of only the target nucleic acid sequence, despite the fact that a generic primer which is complementary to the region of common sequence (and therefore hybridises to both nucleic acid sequences) is used.

The first region of common sequence preferably comprises a length of at least 10 nucleotides, more preferably at least 20 nucleotides.

The first region of common sequence is upstream of a first region of dissimilar sequence. The first region of dissimilar sequence is upstream of a second region of dissimilar sequence. By “upstream” it is meant upstream in terms of the direction of sequencing. The sequencing primer first hybridises to a region comprising at least a portion of the first region of common sequence. As the primer is extended (in the downstream direction) the first region of dissimilar sequence acts as a template for primer extension before the second region of dissimilar sequence. Because primer extension typically proceeds in the 5′ to 3′ direction (as nucleotides are added at the 3′ end of the nascent chain), the first region of common sequence typically lies 3′ to the first region of dissimilar sequence, and the first region of dissimilar sequence typically lies 3′ to the second region of dissimilar sequence.

By “region of dissimilar sequence” it is meant that the sequence of the target nucleic acid is different from the non-target nucleic acid in this region. In one embodiment the first and second regions of dissimilar sequence are contiguous, that is the second region of dissimilar sequence immediately follows the first region of dissimilar sequence with no intervening region of common sequence. In an alternative embodiment, the first and second dissimilar sequences are separated by a second region of common sequence.

In one embodiment the target nucleic acid sequence and the non-target nucleic acid sequence comprises one or more further regions of dissimilar sequence. For instance, there may be a third, fourth, fifth or subsequent regions of dissimilar sequence downstream of the second region of dissimilar sequence. However, there must be at least two regions of dissimilar sequence. Each region of dissimilar sequence is separated by a further region of common sequence. The method permits the determination of the sequence of the target nucleic acid sequence downstream of the second region of dissimilar sequence as far as the sequencing reaction is capable of proceeding.

The length of the first and second regions of dissimilar sequence is not particularly limited. Any length of dissimilar sequence may be used from a single nucleotide upwards. In a preferred embodiment, either or both regions of dissimilar sequence comprises an SNP.

The method comprises a step of contacting a preparation with an oligonucleotide primer complementary to at least a portion of the first region of common sequence. This means that at least a portion of the primer is complementary to a sequence which is present in both the target nucleic acid sequence and the non-target nucleic acid sequence. Thus the primer is capable under suitable conditions (and in the absence of any blocking agent) of hybridising to both the target nucleic acid sequence and the non-target nucleic acid sequence.

In a preferred embodiment, the primer is complementary to a sequence which is found entirely within the first region of common sequence. This means that the hybridisation site of the primer has an identical sequence in both the target and non-target nucleic acid sequence. However, in an alternative embodiment a primer may be used which is capable of hybridising to a sequence a part of which differs between the target nucleic acid sequence and the non-target nucleic acid sequence. In this embodiment, the primer may be fully complementary to a sequence found in either the target or non-target nucleic acid sequence, but a part of the primer may not be complementary to the other nucleic acid sequence. Thus, only a part of the primer is capable of hybridising to one of the nucleic acid sequences. Alternatively, a mixed primer may be used such that the primer contains two species, a first species complementary to the target nucleic acid sequence and a second species complementary to the non-target nucleic acid sequence. The difference in sequence between the target and non-target nucleic acid sequence in the region to which the primer hybridises preferably should be limited to one or two nucleotides, more preferably one nucleotide. The differences should also be located in a region of the nucleic sequences towards which the 5′ end of the primer hybridises. If mismatches are located near the 3′ end of the primer, it is more likely that polymerisation will be inhibited. These embodiments fall within the scope of the invention provided that under the hybridisation conditions employed, the primer is not capable of selectively hybridising only to one of the two nucleic acid sequences. If that were the case, it would be unnecessary to perform a blocking step, because sequencing would proceed only from one of the two nucleic acid sequences.

The nature of the primer is not particularly limited, provided that it is capable of initiating a sequencing reaction when hybridised to a nucleic acid. Preferably the primer is a single-stranded DNA. The length of the primer is preferably 5 to 50 nucleotides, more preferably 10 to 50 nucleotides, more preferably 10 to 40 nucleotides and most preferably 15 to 30 nucleotides. Suitable primers may be designed according to standard techniques known to those skilled in the art for selecting primers for polymerase reactions, such as for sequencing and for amplification of DNA by the polymerase chain reaction (PCR).

The preparation is contacted with the primer, typically by adding an aqueous solution of the primer to a preparation containing a suitable amount of nucleic acid. Hybridisation conditions are then selected so that the primer hybridises to the first region of common sequence of the nucleic acid, according to criteria well known to those skilled in the art. An appropriate temperature and salt content for hybridisation needs to be selected according to the length of the oligonucleotide primer and its G-C content, amongst other things (Old & Primrose (1994), Principles of Gene Manipulation, Blackwell Science and Maniatis et al (1992), Molecular Cloning, A Laboratory Manual, Cold Spring Harbor Laboratory, New York. Typically the hybridisation temperature should be close to the melting temperature (Tₘ) of the primer. Tₘ is defined as the temperature at which the primer and its target are 50% dissociated, and for oligonucleotide primers may be calculated according to the “Wallace rule” by the following formula:

Tₘ=4×(number of G:C base-pairs)+2×(number of A:T base-pairs)

Preferably the hybridisation temperature should be within 2° C. of Tₘ. Accordingly, for a 20-mer oligonucleotide probe with 50% G-C content, the Tₘ is about 60° C. and a suitable hybridisation temperature would be 58° C.

Once the primer is hybridised to the nucleic acid sequence, the preparation is subjected to a sequencing reaction. The sequencing reaction may be any type of nucleic acid sequencing reaction, provided that it involves extension or elongation of the primer when hybridised to a nucleic acid sequence. Primer extension is typically performed using a DNA polymerase, such as Thermus aquaticus or Pfu DNA polymerase for reactions involving a high-temperature step, or other suitable DNA polymerases such as Klenow DNA polymerase where there is no high-temperature step. Preferably the sequencing reaction comprises real-time sequencing, such as pyrosequencing. In another embodiment, the sequencing reaction comprises Sanger sequencing using dideoxynucleotides.

The sequencing reaction proceeds into the second region of dissimilar sequence of the target nucleic acid sequence. Typically this means that at least some of the primer hybridised to the target nucleic acid sequence is extended so that the extended primer contains incorporated nucleotides complementary to one or more nucleotides in the second region of dissimilar sequence of the target nucleic acid. In certain embodiments involving the use of dideoxynucleotide terminator sequencing, only a fraction of the primer may be extended into the second region of dissimilar sequence, as some of the extending primer is terminated at each position in order to determine the sequence.

The method comprises a step of blocking the sequencing reaction between the primer and the non-target nucleic acid. The blocking step prevents the production of sequencing products from non-target nucleic acid, so that in the second region of dissimilar sequence, the only product that is seen is derived from the target nucleic acid sequence. This allows the target nucleic acid sequence to be determined, because the interference from the non-target nucleic acid sequence is removed. The method also allows a particular sequence in the first region of dissimilar sequence to be determined as being associated with a particular sequence in the second region of dissimilar sequence, by intentionally blocking the sequencing reaction when a particular nucleotide is present at the first region of dissimilar sequence.

The blocking step may be performed before the sequencing reaction begins, or after the sequencing reaction has been initiated, provided that substantially no primer has been extended into the second region of dissimilar sequence of the non-target nucleic acid.

The blocking step preferably comprises contacting the preparation with a terminator nucleotide. A terminator nucleotide is a nucleotide which when incorporated into the primer, prevents further extension of the primer. Preferably the terminator nucleotide is a dideoxy nucleotide.

The terminator nucleotide is incorporated into the primer hybridised to the non-target nucleic acid sequence, but not into the primer hybridised to the target nucleic acid sequence. The terminator nucleotide may be incorporated into an unextended primer before the sequencing reaction begins, or may be incorporated into a primer which has been partially extended, provided that the terminator nucleotide is incorporated before the primer has been extended as far the second region of dissimilar sequence. In standard dideoxy terminator sequencing, terminator nucleotides are used which randomly terminate individual primer products at different positions. According to the present invention, a terminator nucleotide is used which terminates only the primer hybridised to the non-target nucleic acid sequence, preferably at a single, predetermined position in all of the primer bound to the non-target nucleic acid sequence. In any case, all of the primer bound to the non-target sequence must be terminated before reaching the second region of dissimilar sequence.

In a preferred embodiment, a primer is used which is complementary to a region of common sequence, wherein the 3′ nucleotide of the primer is complementary to a nucleotide in each nucleic acid which is immediately 5′ to the first nucleotide of the first region of dissimilar sequence. Thus when the primer hybridises to each nucleic acid, it is immediately adjacent to the first region of dissimilar sequence. A terminator nucleotide is then added which is complementary to a first nucleotide at that position in the non-target nucleic acid sequence, but not to a second nucleotide at the corresponding position in the target nucleic acid sequence. Unincorporated terminator nucleotide is then removed, either by washing (especially if the nucleic acid is linked to a solid support) or by the use of a nucleotide-degrading enzyme, such as apyrase. The preparation is then subjected to a sequencing reaction, without allowing the primer to separate from the nucleic acid to which it is bound. In this way, the primer bound to the non-target nucleic acid sequence is terminated, and no sequencing reaction proceeds in respect of the non-target nucleic acid sequence. The target nucleic acid sequence is free to allow primer extension and the sequencing reaction proceeds only in respect of the target nucleic acid sequence.

In a preferred embodiment, the terminator nucleotide is capable of covalently cross-linking the primer to the non-target nucleic acid sequence. Alternatively a primer comprising Peptide Nucleic Acid (PNA) or (L-ribo-)Locked Nucleic Acid (LNA) nucleotides, described in WO 95/15974 and WO 00/66604 respectively, can be used to achieve a higher melting temperature of the primer/nucleic acid sequence complex.

If the primer is covalently linked to the non-target nucleic acid sequence or the primer/nucleic acid sequence complex has a high melting point, a standard sequencing reaction can then be performed. Typically such a sequencing reaction utilises an electronic thermocycler, in order to allow a number of cycles of primer hybridisation to the target nucleic acid sequence, elongation by a polymerase and separation of extended products from the template. Four separate sequencing reactions may be performed, each containing one dideoxy terminator (ddATP, ddCTP, ddGTP or ddTTP) and the products visualised in separate lanes by polyacrylamide gel electrophoresis and autoradiography. If dye terminators comprising fluorescent labels are employed, wherein the labels fluoresce at different wavelengths to indicate each particular terminator nucleotide, a single sequencing reaction can be used.

Alternatively, if the terminator nucleotide is incapable of covalently crosslinking to the nucleic acid (or the primer/nucleic acid sequence complex has a low melting point), it is important to ensure that the terminated primer does not separate from the non-target nucleic acid sequence during the sequencing reaction, as this would allow further unterminated primer to bind to the non-target nucleic acid sequence. Accordingly, in this embodiment, it is preferable to maintain the temperature of the sequencing reaction below the denaturation temperature of the primer/nucleic acid complex. For double-stranded nucleic acids, such as double-stranded DNA, the preparation can first be heated to an elevated temperature, such as 95° C. in order to separate the DNA strands. The preparation is then typically cooled to a suitable hybridisation temperature for the primer (such as 60° C. for a 20-mer oligonucleotide with 50% G-C content). Following addition of the terminator nucleotide and the removal of unincorporated terminator, the sequencing reaction is then performed at a constant temperature (such as room temperature or 37° C.) without thermocycling.

In a preferred embodiment, the sequencing reaction comprises a method of sequencing based on the detection of the release of pyrophosphate. Applicable methods are disclosed in WO 98/28440 and in Science (1998) Vol 281, pages 363 to 365, the contents of which are incorporated herein by reference. Such methods have been termed “pyrosequencing”. According to one suitable pyrosequencing method, the nucleic acid to be sequenced is incubated with the primer, DNA polymerase, ATP sulfurylase, firefly luciferase and a nucleotide-degrading enzyme such as apyrase. Four nucleotides are added stepwise, wherein a nucleotide will only become incorporated into the growing DNA strand and release pyrophosphate (PPi) if it is complementary to the base in the template strand. Any release of PPi is detected enzymically, for example by an enzyme cascade resulting in the production of light which is detected in a suitable light-sensitive device such as a luminometer or a charge-coupled device camera. Unincorporated nucleotides are degraded between each cycle by the nucleotide-degrading enzyme, so that after the first nucleotide has been degraded, the next nucleotide can be added. As this procedure is repeated, longer stretches of the template sequence are deduced.

A method based on the detection of the release of pyrophosphate, involving the stepwise addition of nucleotides and real-time detection of their incorporation, is preferred for performing the sequencing reaction according to the present invention, because it does not require a step of heating which would separate the terminated primer from the non-target nucleic acid sequence. Pyrosequencing is preferably performed using a single-stranded template, which may be suitably prepared by biotin capture of one strand on magnetic beads. The single-stranded template may be free in solution or immobilised on a solid support. Alternatively, a double-stranded DNA template may be employed if the enzymes used in the method are thermostable. In such an embodiment a single heating step is used to denature the double-stranded DNA, followed by a step in which the primer is allowed to anneal. Following the blocking step the extending primer is not separated from its template.

Earlier methods based on the detection of the release of pyrophosphate such as those disclosed in WO 93/23562 and WO 98/13523 are also applicable in the present invention. These methods do not use a nucleotide-degrading enzyme, and therefore require immobilisation of DNA on a solid support and washing steps between each nucleotide addition.

When a method involving the stepwise addition of individual nucleotides (such as pyrosequencing) is used, the primer need not necessarily hybridise immediately adjacent to the first region of dissimilar sequence. Nucleotides can be added stepwise until the first region of dissimilar sequence is reached. At this point, a terminator dideoxynucleotide can be added which is complementary to a nucleotide at this position in the non-target nucleic acid sequence. The terminator is not complementary to a nucleotide at the corresponding position in the target nucleic acid sequence. The “corresponding position” is determined relative to the primer binding site or the start of the first region of dissimilar sequence. Because the primer hybridises to an identical sequence in both the target and non-target nucleic acid sequences, a corresponding position in the target nucleic acid sequence may be defined as being a particular number of nucleotides downstream of the last nucleotide of the primer binding site, or a particular number of nucleotides downstream of the first nucleotide of the first region of dissimilar sequence. The terminator will then become incorporated only into the non-target nucleic acid sequence and prevent further elongation from this sequence. The first nucleic sequence can continue to be sequenced in the same way without interfering signal from the other nucleic acid.

In a preferred embodiment of the present invention, the method involves determining the combination of individual SNPs which exist in a particular region on one chromosome of a pair in a subject. Determining the association of alleles such as SNPs is termed haplotyping. In this embodiment, each of the first and second regions of dissimilar sequence comprise a single nucleotide. The target nucleic acid sequence comprises a particular locus (such as a particular gene, part of a gene or regulatory element) on one chromosome of a pair in the individual subject, and the non-target nucleic acid sequence comprises the corresponding sequence on the other chromosome in the pair. The locus comprises two or more SNPs. The first and second regions of common sequence comprise parts of the locus which are non-polymorphic between the two chromosomes.

Where the method is used to determine associations of previously identified SNPs in a subject sample, one of the known alleles for the first SNP is used to block further sequencing from that chromosome. In this way, further sequencing proceeds only from the other chromosome; the base present in the second SNP is determined for the other chromosome and the combination of SNPs present on each chromosome can be determined.

For example, two alleles A (on chromosome A) and C (on chromosome A′) for SNP-1 and two alleles G and T for SNP-2 may be known to be present within a particular gene in a subject, but the combination of alleles on each chromosome (haplotype) is unknown. The possible haplotypes (for chromosome A and its pair chromosome A′) for this individual are therefore either (1) A-G (on chromosome A) and C-T (on chromosome A′), or (2) A-T and C-G. In order to distinguish between these possibilities, dideoxyguanosine triphosphate is added to the preparation so that it becomes incorporated into the chromosome A′ which bears a C at SNP-1. Sequencing then proceeds only on chromosome A. If the sequencing results indicate a G at SNP-2, then (1) is correct. When dideoxythymidine triphosphate is added for incorporation at SNP-1, a T would be expected at SNP-2.

HLA genotyping is one area where haplotyping is particularly useful. Genotyping of the two haplotypes of the HLA genes is crucial to the success of the transplantation of organs and bone marrow. In a preferred embodiment, the locus comprises a human Class I or Class II HLA gene.

The invention will now be described by way of example only, with reference to the following specific drawings.

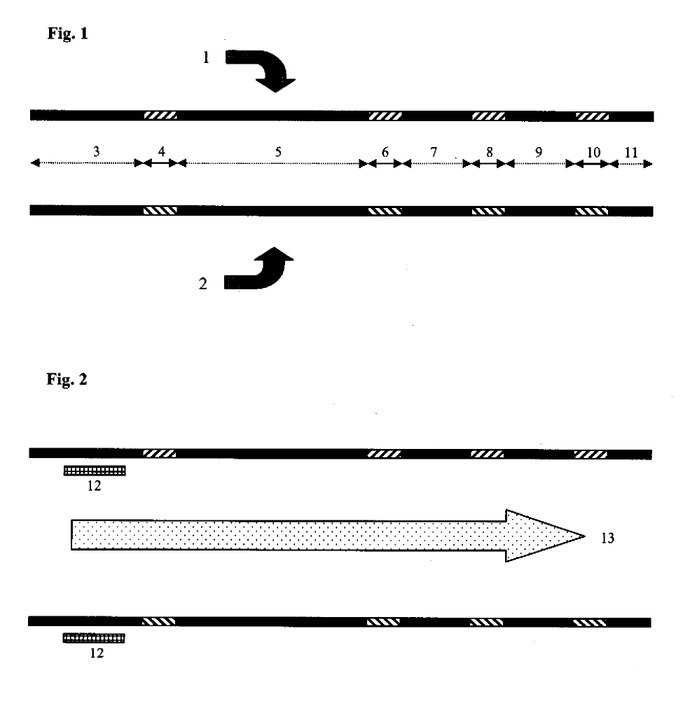

FIG. 1 shows a target nucleic acid 1 and a non-target nucleic acid 2. The target nucleic acid and non-target nucleic acid each have a first region of common sequence 3, a first region of dissimilar sequence 4 and a second region of dissimilar sequence 6. In the embodiment shown, a second region of common sequence 5 lies between the first and second regions of dissimilar sequence. Third and fourth regions of dissimilar sequence (8 and 10) and third, fourth and fifth regions of common sequence (7, 9 and 11) are also shown.

FIG. 2 shows an oligonucleotide primer (12) which is complementary to the first region of common sequence and which hybridises thereto. A sequencing reaction proceeds in the direction of the arrow 13, such that the primer 12 is extended in the direction of the arrow using the target and non-target nucleic acid sequences as a template.

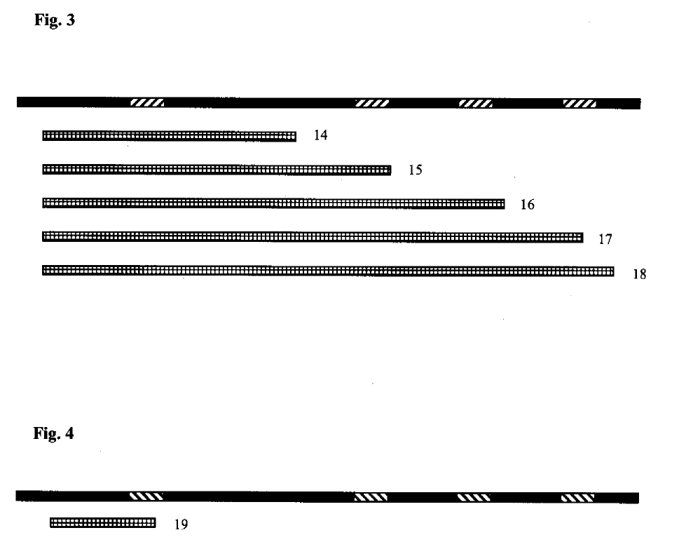

FIG. 3 shows sequencing reaction products (14 to 18) resulting from extension of the primer using the target nucleic acid as a template. The sequencing reaction proceeds at least as far as the second region of dissimilar sequence.

FIG. 4 shows a sequencing reaction product (19) resulting from extension of the primer using the non-target nucleic acid as a template. The sequencing reaction does not proceed as far as the second region of dissimilar sequence.

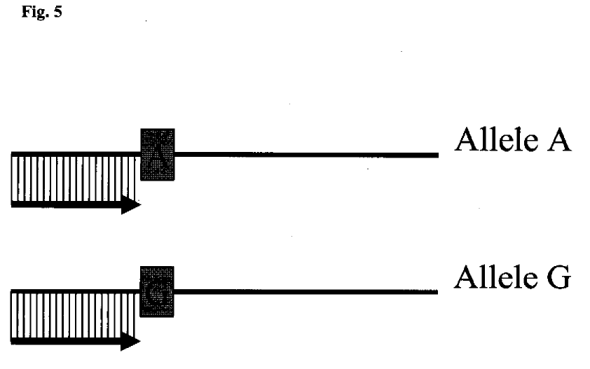

FIG. 5 shows a primer hybridising to a target nucleic acid sequence (Allele A) and to a non-target nucleic acid sequence (Allele G). The primer hybridises adjacent to an SNP at which the sequence of alleles A and G differ.

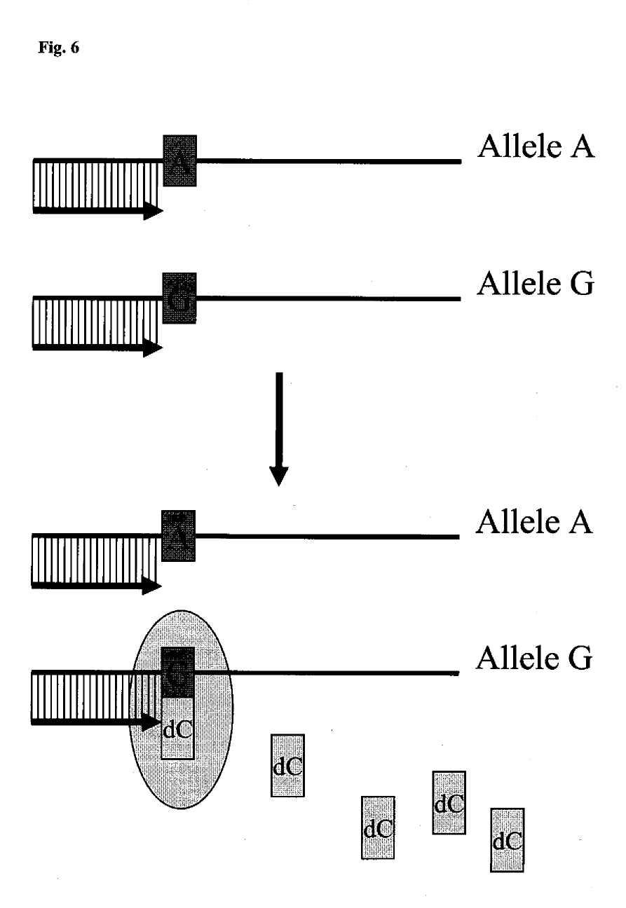

FIG. 6 shows the addition of dideoxycytosine which becomes incorporated into the primer bound to allele G in the presence of DNA polymerase.

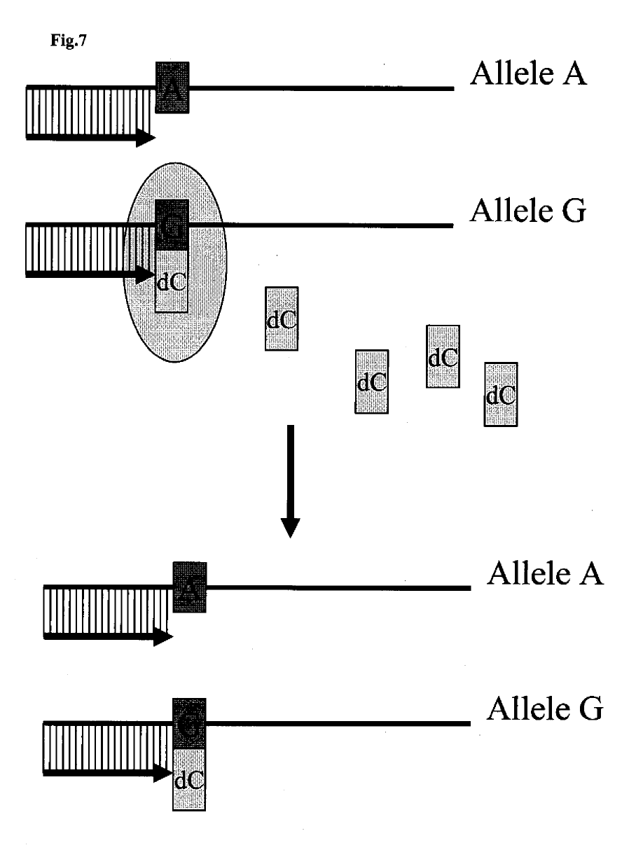

FIG. 7 shows the subsequent removal of incorporated dideoxycytosine.

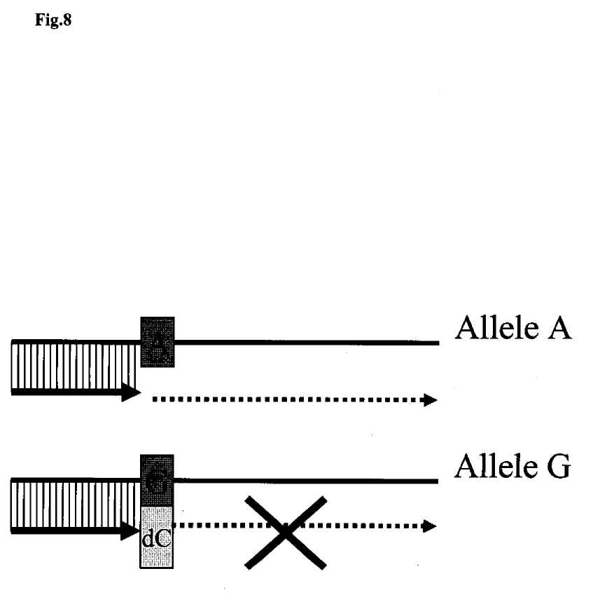

FIG. 8 shows that when the preparation is subjected to a sequencing reaction, the primer bound to allele A is extended but the primer bound to allele G is not extended.

1. A method for determining a target nucleic acid sequence, the method comprising the steps of:
(a) contacting a preparation comprising the target nucleic acid sequence and a non-target nucleic acid sequence, the target nucleic acid sequence and the non-target nucleic acid sequence each having a first region of common sequence upstream of a first region of dissimilar sequence upstream of a second region of dissimilar sequence, with an oligonucleotide primer complementary to at least a portion of the first region of common sequence, under conditions to hybridise the primer thereto; and
(b) subjecting the resulting preparation to a sequencing reaction, such that the sequencing reaction proceeds into the second region of dissimilar sequence of the target nucleic acid sequence, thereby determining at least the second region of dissimilar sequence of the target nucleic acid sequence; and
wherein the method further comprises a step of blocking the sequencing reaction between the primer and the non-target nucleic acid sequence, such that the sequencing reaction does not proceed into the second region of dissimilar sequence of the non-target nucleic acid sequence.

2. The method according to claim 1, wherein the target nucleic acid sequence and the non-target nucleic acid sequence each have a second region of common sequence which lies between the first and second regions of dissimilar sequence.

3. The method according to claim 1, wherein the blocking step comprises contacting the preparation with a terminator nucleotide, under conditions to incorporate the terminator nucleotide into the extended or unextended primer hybridised to the non-target nucleic acid sequence but not into the extended or unextended primer hybridised to the target nucleic acid sequence.

4. The method according to claim 3, wherein the conditions are such that the terminator nucleotide is incorporated into substantially all of the extended or unextended primer hybridised to the non-target nucleic acid sequence, before the sequencing reaction reaches the second region of dissimilar sequence.

5. The method according to claim 4, wherein contacting the preparation with the terminator nucleotide is after step (a) and before step (b) of claim 1.

6. The method according to claim 4, wherein the terminator nucleotide is complementary to a first nucleotide comprised in the first region of dissimilar sequence of the non-target nucleic acid sequence, but the terminator nucleotide is not complementary to a second nucleotide at a corresponding position in the target nucleic acid sequence.

7. The method according to claim 3, wherein the terminator nucleotide is a dideoxy nucleotide.

8. The method according to claim 7, wherein the terminator nucleotide is capable of covalently cross-linking the primer to the non-target nucleic acid.

9. The method according claim 1, wherein the second region of dissimilar sequence comprises a single nucleotide.

10. The method according to claim 1, wherein the first region of dissimilar sequence comprises a single nucleotide.

11. The method according to claim 1, wherein the sequencing reaction comprises a method of sequencing based on detection of released pyrophosphate.

12. The method according to claim 11, wherein the sequencing reaction comprises pyrosequencing.

13. The method according to claim 1, wherein said preparation comprises DNA derived from two or more subjects.

14. A method for determining a plurality of target nucleic acid sequences which method comprises the steps of:
(a) contacting a preparation wherein the plurality of target nucleic acid sequences is comprised in a preparation comprising a plurality of target nucleic acid sequences and a plurality of corresponding non-target nucleic acid sequences, wherein each target nucleic acid sequence in the preparation corresponds to one or more corresponding non-target nucleic acid sequences in the preparation, each target nucleic acid sequence and each corresponding non-target nucleic acid sequence has a first region of common sequence upstream of a first region of dissimilar sequence upstream of a second region of dissimilar sequence, the first region of common sequence of each target nucleic acid sequence is the same as the first region of common sequence of its corresponding non-target nucleic acid sequences, the first region of dissimilar sequence of each target nucleic acid sequence is different to the first region of dissimilar sequence of its corresponding non-target nucleic acid sequences, the second region of dissimilar sequence of each target nucleic acid sequence is different to the second region of dissimilar sequence of its corresponding non-target nucleic acid sequences, with a plurality of oligonucleotide primers, wherein each primer is complementary to at least a portion of the first region of common sequence of a target nucleic acid sequence and its corresponding non-target nucleic acid sequence, under conditions to hybridise the primer thereto; and
(b) subjecting the resulting preparation to a sequencing reaction, such that the sequencing reaction proceeds into the second region of dissimilar sequence of the target nucleic acid sequences, thereby determining at least the second region of dissimilar sequence of each target nucleic acid sequence; and wherein the method further comprises a step of blocking the sequencing reaction between each primer and each corresponding non-target nucleic acid sequence, such that the sequencing reaction does not proceed into the second region of dissimilar sequence of each corresponding non-target nucleic acid sequence.

15. The method according to claim 1, wherein the target nucleic acid sequence and the non-target nucleic acid sequence comprise one or more further regions of dissimilar sequence downstream of the second region of dissimilar sequence.

16. The method for determining the haplotype of a subject from a sample comprising DNA from the subject, comprising a method as defined in claim 1, wherein the preparation comprises the sample, the target nucleic acid sequence includes a locus on a first chromosome of a pair of chromosomes, the non-target nucleic acid sequence comprises the corresponding locus on the second chromosome of the pair, the locus comprising two or more single nucleotide polymorphisms for which the subject is heterozygous, wherein the sequencing reaction is conducted to determine the sequence of the locus on the first chromosome of the pair thereby determining the haplotype of the subject.

17. The method according to claim 16, wherein the locus comprises a human Class I or Class II HLA gene.

18. A method of pyrosequencing a sample of DNA from a subject for determining the haplotype of the subject comprising the steps of: pyrosequencing a target locus on a first chromosome of a pair, the target locus comprising two or more single nucleotide polymorphisms, and blocking from sequencing the corresponding locus on the second chromosome of the pair.

19. The method according to claim 18, wherein the corresponding locus on the second chromosome of the pair is blocked from sequencing by incorporation of a terminator nucleotide into an oligonucleotide primer hybridised to the second chromosome.

20. A kit for determining one or more target nucleic acid sequences, wherein the one or more target nucleic acid sequences are comprised in a preparation comprising one or more target nucleic acid sequences, one or more non-target nucleic acid sequences, the one or more target nucleic acid sequences and the one or more non-target nucleic acid sequences each having a first region of common sequence upstream of a first region of dissimilar sequence upstream of a second region of dissimilar sequence, comprising one or more oligonucleotide primers complementary to at least a portion of the first region of common sequence and one or more terminator nucleotides.

21. The kit according to claim 20, wherein the terminator nucleotide comprises a dideoxy nucleotide.

22. The kit according to claim 21, wherein the kit includes dideoxy-ATP, dideoxy-CTP, dideoxy-GTP and/or dideoxy-TTP.

23. The kit according to claim 20, further comprising deoxy-ATP, deoxy-CTP, deoxy-GTP, deoxy-TTP, a DNA polymerase, ATP sulfurylase, firefly luciferase and/or a nucleotide-degrading enzyme.
